(19) United States
(12) Patent Application Publication
(10) Pub. No.: US 2007/0191711 A1
(43) Pub. Date: Aug. 16, 2007

(54) Liquid processing and handling apparatus and associated method for use in medical procedures

(75) Inventors: Christopher Bush, Commack, NY (US); Scott Isola, Deer Park, NY (US); Ronald R. Manna, Valley Stream, NY (US)

Correspondence Address: COLEMAN SUDOL SAPONE, P.C., 714 COLORADO AVENUE, BRIDGE PORT, CT, 06605-1601, US

(73) Assignee: MISONIX, INCORPORATED, Farmingdale, NY (US)

(21) Appl. No.: 11/355,055

(22) Filed: Feb. 15, 2006

(51) Int. Cl. A61B 8/00

(52) U.S. Cl. 600/437

(57) ABSTRACT

In preparation for a medical procedure utilizing a medical instrument such as a high intensity focused ultrasound probe sterile water is gravity fed to a reservoir container through a hydrophobic hollow fiber or membrane filter connected to a vacuum pump, whereby the reservoir container is filled with degassed sterile water. The reservoir is operatively connected to the medical instrument in a hydraulic circuit through which the degassed sterile water is pumped from the reservoir container. The circuit is purged of air and then closed to render the medical instrument in condition for a medical procedure.

### BACKGROUND OF THE INVENTION

This invention relates to a scheme of providing sterile, degassed water for surgical procedures, particularly those involving High Intensity Focused Ultrasound (HIFU) devices and the handling equipment and techniques necessary for their production and use.

High Intensity Focused Ultrasound devices for use in various surgical procedures have been described in medical literature since the late 1940's. These devices use the same type of energy source as is found in SONAR transmitters or more recently, Diagnostic Ultrasound Scanners. However, instead of transmitting these waves through the body as a collimated beam, HIFU transmitters instead focus the acoustic energy to a theoretical point distal from the transducer/tissue interface much the same as a magnifying glass focuses light beams. The point at which the acoustic energy intensity is greatest is called the focal point. If the energy intensity is great enough at this point, the tissue will begin to heat. As the energy level is increased further, the tissue will heat to the point where cell death occurs, called the necrosis point. After this, the tissue is unviable and will die, even if the energy source is turned off. In this manner, tissue can be destroyed deep inside the body without disturbing the intervening tissue, where the acoustic intensity is below that where necrosis will occur.

There have been several applications or implementations of this theory described in the prior art. For instance, U.S. Pat. Nos. 5,054,470, and 4,955,365 describe hardware and methods for the use of this energy and the advantages gained through its application. However, almost all of these embodiments have the requirement of creating an acoustically efficient coupling between the acoustic wave generator and the tissue itself.

In diagnostic ultrasound devices, such as fetal monitors or Doppler Cardiac Monitors, the transducer face is placed directly against the skin of the subject. For these devices, a silicone gel or paste is employed to give a good acoustic coupling from the transducer face to the skin itself. This gel serves to lubricate the surface so that the transducer may be rubbed on the skin without binding. The gel fills the voids between the skin and the transducer face to eliminate air gaps and also serves to cool the interface so as to not induce friction or acoustic burning. Several embodiments of HIFU devices use a moving piezoelectric transducer face to aim the focal point at different targets within the body. The transducer is moved by stepping motors with digital feedback under control of the main computer. The transducer is placed within a hollow sleeve with an opening through which the acoustic beams emanate. The transducer is free to move longitudinally and rotationally without touching the skin at all. This assembly is called the transducer head, FIG. 1.

Since gas presents infinite impedance to acoustic energy, no energy would flow from the transducer face to the body organ in contact with the transducer head if the internal volume remains filled with air. In current embodiments, a flexible membrane is placed over the transducer head and sealed. The internal volume of the head is filled with water that provides acoustic coupling between the piezoelectric transducer and the body organ itself. Since most mammalian bodies are water based, the acoustic impedance between the transducer coupling water and the body is low, thereby providing efficient transmission of the acoustic waves from the piezoelectric element and the target tissue.

The water used for this coupling must have special properties when compared to potable water supplies. If the water is to be used under the skin, it must be sterile to an SAL of 10⁻⁶. Moreover, the water must be chemically compatible with the body and be free of pathogens and foreign matter. In general, then, the water must pass the standards as set down in the United States Pharmacopeia (USP) for either Water for Injection or Water for Irrigation.

In addition to the requirements of the USP, the water used as a coupling agent for HIFU must also be gas free. As stated, gas presents as high impedance to acoustic waves. If a large amount of gas is entrained in the liquid, the bulk impedance of the liquid rises. This causes absorption of the acoustic waves in the fluid, reducing the amount of energy being transmitted to the body and potentially heating the water to the point of burning the tissue in contact with the transducer head. In addition, most HIFU devices incorporate diagnostic ultrasound devices in order to view the internal features of the body and aid in targeting tissue. When gases are present in the water, the diagnostic image is degraded, sometimes to the point where it becomes unreadable. It has been found that water which as been degassed to a level below 4 ppm is optimal for use in surgical HIFU procedures that will breach the skin barrier.

Therefore it is required that a supply of water which is pure to the standards of USP Water for Irrigation as well as degassed to a level of less than 4 ppm be readily available and be economical for single use in an surgical environment.

The combination these three conditions simultaneously present a hurdle when obtaining the water. Water for medical irrigation purposes is readily available and inexpensive, but all such water is not degassed to a level where it can be used in HIFU procedures. If that water is to be degassed on site, it will be rendered unsterile and therefore unusable. Since HIFU technology is emerging and the number of HIFU procedures each year is relatively low as a result, sterile, degassed water when obtained through traditional commercial channels is expensive and presents problems in shipping long distances.

Another issue in some HIFU systems is that the liquid transport system, including reservoirs, must be of constant volume. No expansion of the system is permitted, such as would be the case with a flexible bag used as the main reservoir. All reservoirs must be of rigid plastic or glass construction; with the tubing being a semi rigid plastic design. The reason for this is that the flexible membrane over the transducer head aperture will expand or contract as the liquid pressure changes. This could occur when the height of a probe head 12 is changed relative to that of a main reservoir 60 (FIGS. 2A and 2B). By application of Bernoulli's equations, those skilled in the art will appreciate that the pressure head of fluid will go up as the vertical distance between the reservoir 60 and the probe head 12 increases and conversely, will be less as the vertical distance between them decreases (compare FIGS. 2A and 2B). As these changes occur, the dimension of a bolus 62 will increase or decrease accordingly. This bolus must be of constant height during the procedure in order not to affect the targeting accuracy of the system.

It is therefore desired to create sterile, degassed water on site in operating rooms around the world at an economical price so patient safety, product specification and economic goals are met. In addition, it is desired that a fluid pathway of constant volume be created at the same time.

### OBJECTS OF THE INVENTION

One object of the present invention is to provide hardware and a method of use to allow clinicians to create an ample supply of sterile, degassed water at the point of use in an economical manner.

Another object of this invention is to describe a fluid circuit that will provide a finite volume to allow pressurization of the fluid column for bolus adjustment.

It is another object of this invention to provide a liquid handling system which by its nature does not trap gas bubbles or allows for any air bubbles which are contained in the system to be easily removed.

It is a further object of this invention to provide a system that will not be affected by the relative height differences between fluid reservoirs and a HIFU probe.

It is another object to provide a system that may be cleaned and sterilized such that all components may be located in the sterile field of the operating room.

These and other objects of the invention will be apparent from the drawings and descriptions herein. Although every object of the invention is attained in at least one embodiment of the invention, there is not necessarily any embodiment which attains all of the objects of the invention.

### SUMMARY OF THE INVENTION

A method for use in preparation for a medical procedure that utilizes a medical instrument such as a high intensity focused ultrasound probe comprises, in accordance with the present invention, connecting the instrument to a hydraulic circuit including a pump and a reservoir container, filling the container with degassed sterile irrigation water, removing air from the circuit, and pumping degassed sterile irrigation water from the reservoir container through the instrument and back to the reservoir container.

More particularly, a method for use in preparation for a medical procedure utilizing a medical instrument such as a high intensity focused ultrasound probe comprises (a) feeding sterile water to a reservoir container, (b) during the feeding of the water to the reservoir container, degassing the water, (c) upon a filling of the reservoir container with the degassed sterile water, operatively connecting the reservoir to the medical instrument in a hydraulic circuit, (d) subsequently pumping degassed sterile water from the reservoir container through the circuit, (e) during the pumping of the water, removing air from the circuit, and (f) closing the circuit. Following this procedure creates a closed circuit containing degassed sterile water and having a substantial absence of air, which renders the medical instrument in condition for a medical procedure.

Pursuant to another feature of the present invention, the degassing of the sterile water comprises feeding the sterile water through a degassing hydrophobic hollow fiber or membrane filter and, during that feeding, operating a vacuum pump connected to the hydrophobic hollow fiber or membrane filter to extract dissolved gas from the water. The hydrophobic hollow fiber or membrane filter is operatively disconnected from the reservoir container after the filling thereof, for instance, by actuating a three-way valve to block communication between the hydrophobic hollow fiber or membrane filter and the reservoir container and simultaneously to operatively connect the reservoir container to the medical instrument. An alternative procedure would be to totally remove the hydrophobic hollow fiber or membrane filter from the circuit.

The vacuum pump may be connected to the hydrophobic hollow fiber or membrane filter prior to the feeding of the sterile water through the filter, together with a hydrophobic secondary filter disposed between the vacuum pump and the hydrophobic hollow fiber or membrane filter.

In a particular embodiment of the present invention, the feeding of sterile water to the reservoir container is carried out as a gravity feed operation. Thus, a source of sterile water is connected to an inlet of the hydrophobic hollow fiber or membrane filter and disposed at a vertical elevation higher than that of the hydrophobic hollow fiber or membrane filter, which in turn is disposed at a higher elevation than that of the reservoir container. It is to be noted that once air is removed from the circuit containing the filled reservoir container and the medical instrument and the circuit is then closed, the vertical position of the medical instrument relative to the reservoir container may be altered without affecting the bolus or pressure head in the medical instrument, provided that the hydraulic circuit remains closed.

Pursuant to a further feature of the present invention, the method also comprises connecting an overflow container to the reservoir container and conveying degassed sterile water from the reservoir container to the overflow container. In a gravity feed arrangement, the overflow container is disposed at a vertical location higher than a vertical location of the reservoir container and lower than a vertical location of a source of the sterile water. The overflow container may be provided with an air outlet port impermeable to a passage of liquid water, so that the method further comprises connecting the overflow container to the reservoir container so as to enable a passage of air from the reservoir container to the overflow container and out of the overflow container through the outlet port.

Pursuant to an additional feature of the present invention, the method further comprises connecting to the reservoir container a syringe holding an amount of degassed sterile water, for enabling an increase in water content of the hydraulic circuit after the removing of air from the circuit and after the closing of the circuit. The syringe may be filled with the amount of degassed sterile water from the overflow container by connecting the syringe to the overflow container after a disconnecting or isolating of the overflow container from the reservoir container.

A kit for use in preparation for a medical procedure utilizing a medical instrument such as a high intensity focused ultrasound probe comprises, in accordance with the present invention, a hydrophobic hollow fiber or membrane filter, a reservoir container, a plurality of valves, and tubing for constructing a first hydraulic circuit including the hydrophobic hollow fiber or membrane filter and the reservoir container and a second hydraulic circuit including the reservoir container and the medical instrument. The tubing serves in part to connect an input end of the hydrophobic hollow fiber or membrane filter to a source of sterile medical water in the first hydraulic circuit and to connect a pump between the reservoir container and the medical instrument in the second hydraulic circuit. The tubing also serves to couple the filter to a vacuum pump to degas water passing through the filter in the first hydraulic circuit and to enable a release of air from the second hydraulic circuit during an operation of the pump circulating degassed sterile water from the reservoir container to the medical instrument and back to the reservoir container. The valves are disposed in the first hydraulic circuit so as to facilitate an operative connecting of the filter to the reservoir container to enable flow of degassed sterile water from the filter to the reservoir container and simultaneously an isolating of the medical instrument from the reservoir container and the filter and to enable a subsequent isolation of the filter from the reservoir container while permitting fluid communication between the reservoir container and the medical instrument.

The kit may further comprise an overflow container connectable to the reservoir container via the tubing. The overflow container may be provided with an air outlet port impermeable to a passage of liquid water, the overflow container being connectable to the reservoir container so as to enable a passage of air from the reservoir container to the overflow container and out of the overflow container through the outlet port.

The kit may additionally comprise a syringe connectable to the circuit for containing an amount of degassed sterile water to enable an increase in water content of the hydraulic circuit after a removing of air from the circuit and after a closing of the circuit.

A hydraulic circuit assembly for use in preparing for and carrying out a medical therapeutic method using a medical instrument comprises, in accordance with the present invention, (1) a hydrophobic hollow fiber or filter connectable on an upstream side to a source of sterile medical water and also connectable to a vacuum pump for degassing sterile water flowing through the filter, (2) a three-way valve, (3) a reservoir container operatively coupled to an outlet of the filter via the three-way valve, and (4) a pump operatively linked on an upstream side to the three-way valve and on a downstream side to an inlet of the medical instrument. The probe has an outlet operatively connected to the reservoir container. The hydraulic circuit assembly further comprises (5) at least one valve or outlet port connected to the reservoir container for enabling a removal of air from the reservoir container and from a subcircuit including the reservoir container and the medical instrument.

The circuit assembly may further comprise an overflow container operatively connected to the reservoir container for receiving degassed sterile water therefrom. In that case, the valve or outlet port is provided on the overflow container.

The circuit assembly may also comprise a syringe connectable to the subcircuit including the filled reservoir container and the medical instrument for containing an amount of degassed sterile water to enable an increase in water content of the subcircuit after a removing of air from the subcircuit and after a closing thereof.

### BRIEF DESCRIPTION OF THE DRAWING

FIG. 1 is a schematic elevational view of a typical HIFU transducer head assembly.

FIG. 2 is a diagram of a HIFU transducer head bolus.

FIG. 2A is a graph showing bolus height as a function of liquid level changes.

FIG. 3 is a diagram of a hydraulic circuit in accordance with the present invention.

FIG. 4 is a diagram of a portion of the circuit of FIG. 3, constituting a liquid degassing and reservoir subcircuit.

FIG. 5 is a diagram of another portion of the circuit of FIG. 3, constituting a HIFU transducer head purging subcircuit.

FIG. 6 is a diagram of a HIFU transducer liquid circuit configured from the circuit of FIG. 3.

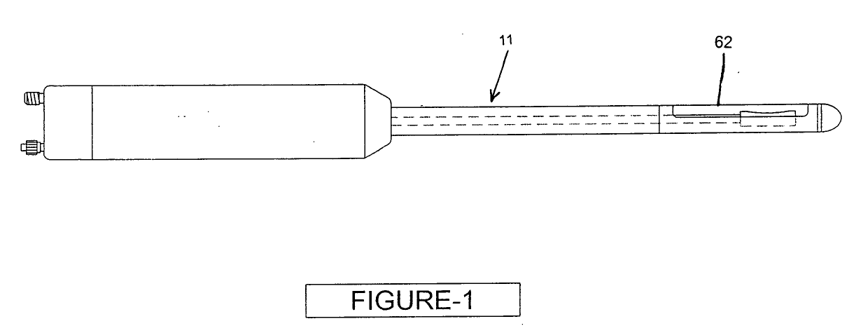

### DETAILED DESCRIPTION

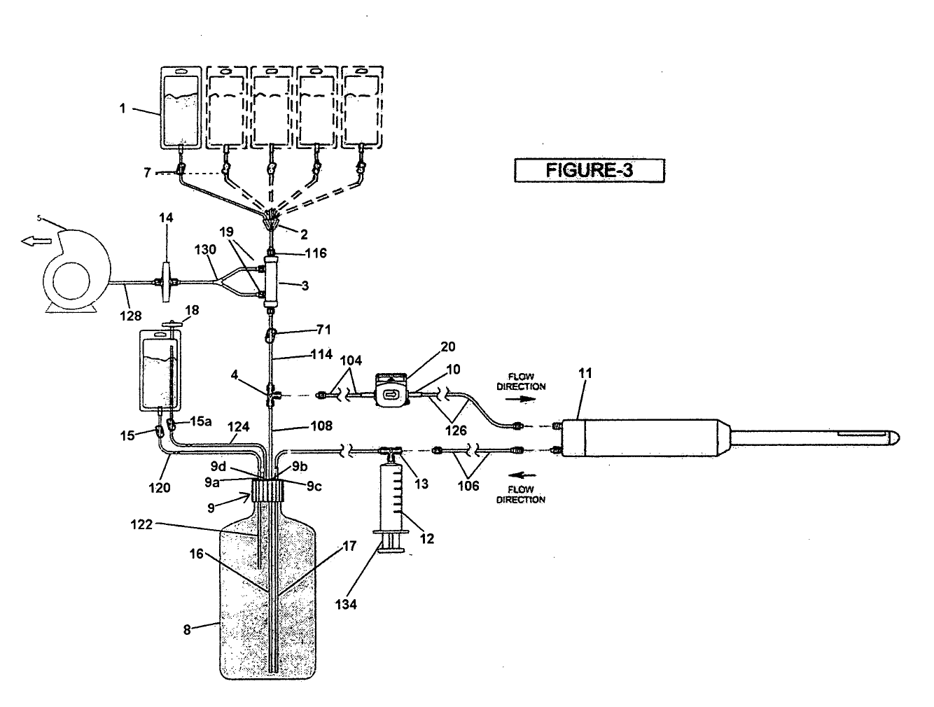

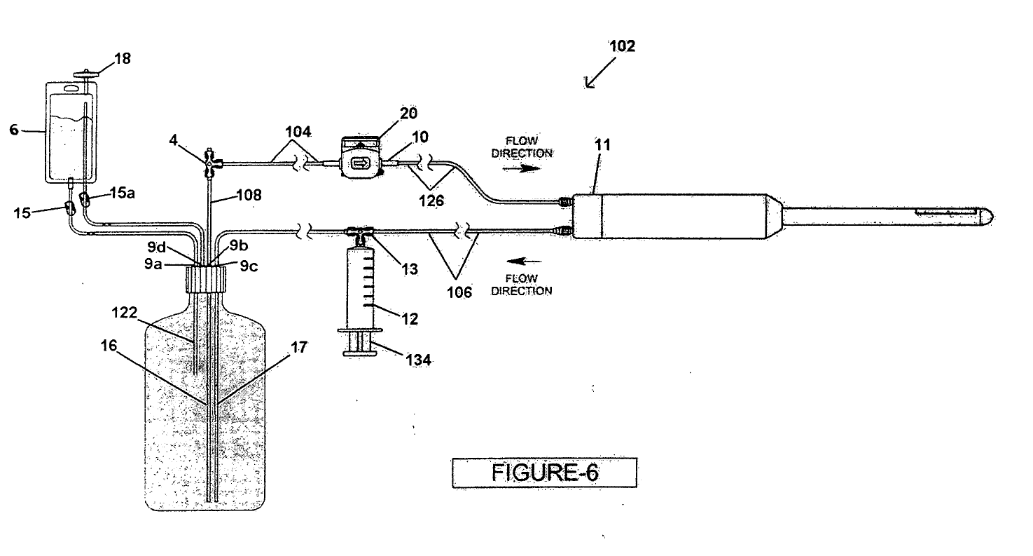

A liquid processing and transport mechanism for a medical instrument such as a high intensity focused ultrasound probe 11 is shown in FIG. 3. In this embodiment, the objective is to construct and load a closed loop pumping system or powered hydraulic circuit 102 (FIG. 6) including the ultrasound probe 11 and a reservoir bottle 8 containing degassed sterile medical irrigation water. More specifically, the closed loop hydraulic circuit 102 includes a liquid manifold such as a three-way valve 4, pump feed tubing 104, a peristaltic pump 20, a pump outlet tube 10, the transducer head assembly or ultrasound probe 11, tubing 106 extending from the probe to reservoir bottle 8, the reservoir bottle 8, and a return tube 108. Hydraulic circuit 102 is loaded with degassed sterile medical irrigation water via an auxiliary hydraulic circuit 110 including one or more sterile water supply bags 1, a bag-to-degasser unit tube 112, a valve or manifold 2, a degasser unit 3, a degasser-to-manifold tube 114, and an overflow bag 6. A top-up syringe 12 may also be part of the system.

All interior and exterior surfaces of the components of the system must be sterilized prior to assembly by steam autoclave, Ethylene Oxide Gas (ETO), gamma irradiation or other means as may be appropriate.

The operating room set-up personnel will assemble the system in the configuration as shown in FIG. 3 using standard luer fittings or other such means of liquid and airtight connections. The one or more sterile water supply bags or bottles 1 serve as a liquid source and may be standard “Water for Irrigation” containers, whether flexible or rigid. As is known in the art, sterile pure water must be used where any portion of the probe 11 is placed in the body under the skin. Such water is readily available in the marketplace and is relatively inexpensive. If a greater volume of liquid is desired than is contained in a single unit, multiple bags or bottles 1 are connected via multi-inlet manifold 2 using standard IV spikes to connect to the sterile water containers. The water supply container or containers 1 must be mounted higher than rigid reservoir container 8 to allow a gravity head to be developed or, alternatively, a pump may be employed. Each water supply bag or bottle 1 should incorporate either a stopcock or a pinch valve 7 to control outlet flow or the tubing attached to each supply bag or bottle 1 should contain a stopcock or shut-off valve.

A liquid tube 112 from a single supply bag or bottle 1 and additionally manifold 2 if multiple bags or bottles 1 are used is then attached to a liquid inlet 116 of degasser unit 3. A degassing unit 3 of sufficient size to accommodate the volumetric flow rate desired is needed to degas the sterile fluid to the required ppm level. Degassing unit 1 is typically a hydrophobic hollow fiber or membrane filter cartridge arranged in a cross flow configuration (tangential flow) and having a molecular weight cut off (or pore size) such that only dissolved gasses pass from the fluid stream when vacuum is applied to on side of the fiber or membrane. When properly specified and used, these units can degas fluids to below 3 ppm dissolved gas at substantial flow rates. These devices are well known to the art and will not be discussed further here.

Liquid outlet tube 114 of degasser unit 3 is connected via manifold or three-way valve 4 and return tube 108 to a cap fitting 9b of reservoir container 8. Pump feed or inlet tubing 104 is likewise connected to a third port of three-way valve, 4, with the other end connected to the inlet of the pump 20.

Overflow container or bag 6 is connected to a cap fitting 9a of reservoir container 8 via a respective tubing run 120. This fitting has a downcomer 122 which projects approximately halfway down into container 8. Overflow container 6 must be mounted higher than rigid reservoir container 8, but lower than sterile water supply bags or bottles 1. A vent line 124 is connected between an upper end of overflow container 6 and a fourth cap fitting or connection 9d on reservoir container 8. This vent line or connection does not include a downcomer in reservoir container 8, in order to allow entrained gas to escape.

Peristaltic pump 20 is provided to force fluid through HIFU probe 11 during a surgical operation. Outlet tube 10 of pump 20 is connected to the liquid feed inlet (not labeled) of probe 11 via a tube 126. It is to be noted that the tube run including the pump inlet tube 104, an internal pump tube (not shown), the pump outlet tube 10, and the connector tube 126 may be constituted by a single unitary length of tubing.

The outlet fitting (not separately designated) of probe 11 is connected to a third opening or cap fitting 9c of reservoir container 8 via tube 106. A three way valve or stopcock 13 is provided in this tubing run 106 to accept a luer fitting of syringe 12, which may be a common off-the-shelf component.

Degasser unit 3 incorporates one or more fittings 19 for enabling connection of the degasser unit to a vacuum pump 5. In this embodiment, the degasser vacuum fittings are connected via tubing 128 to vacuum pump 5 either directly or via a manifold 130. A hydrophobic filter 14 may be installed to prevent liquid transport to vacuum pump 5 in the event of a degasser unit failure.

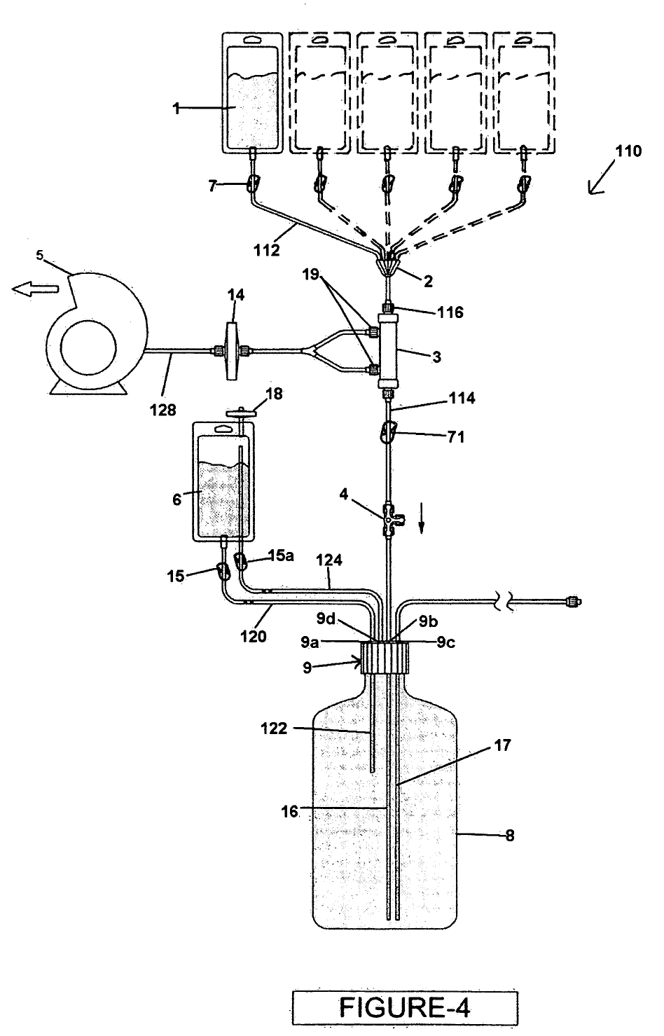

To begin operation, three-way valve 4 is set to flow liquid from sterile water supply bags 1 to rigid reservoir container 8. At least one shut-off valve 7 is opened, as is a pinch clamp connected to liquid outlet tube 118 of degasser unit 3. Shut off valves or pinch clamps 15 and 15a connected to tubes 120 and 124 are likewise opened. Three-way valves 4 and 13 are set to block flow to probe 11. Vacuum pump 5 is then turned on. This effectively creates a liquid charging system or hydraulic circuit 110 as shown in FIG. 4.

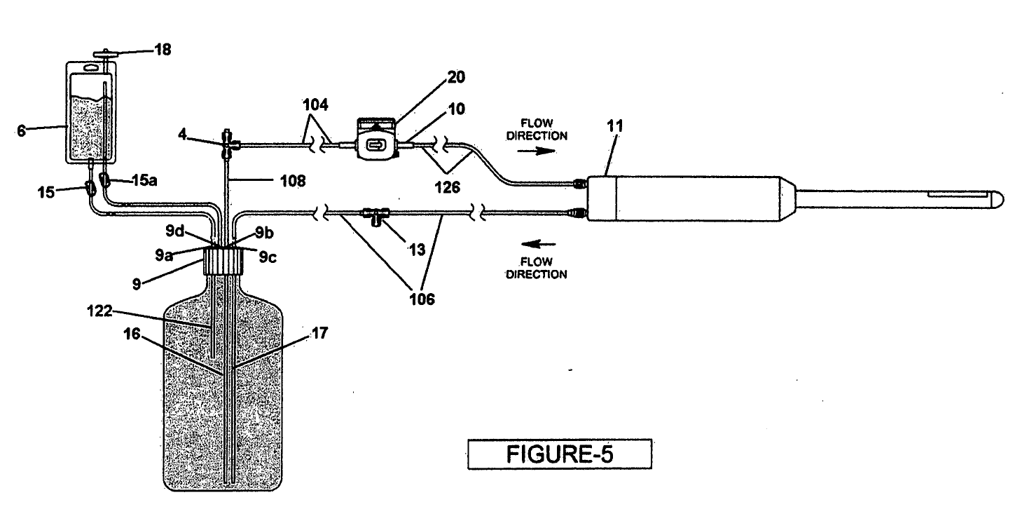

Liquid now flows under gravity head (or is alternatively pumped) through degasser unit 3. The liquid will be degassed and then flow into rigid container 8. A cap 9 of container 8 incorporates rigid downcomers 16 and 17 on fittings 9b and 9c, respectively, to effectively move the outlets of the fittings near the bottom of the reservoir container 8. Flow is maintained until container 8 is totally filled and liquid rises into overflow container 6 through downcomer 122, fitting 9a, and tube 120 by gravity head. Overflow container 6 contains a hydrophobic vent filter 18 to allow air to escape but to block liquid flow. This vent filter 18 permits air to vent from the system during filling so as to create a self air bleeding system. Once the sterile water supply bags 1 are empty or overflow bag 6 is completely full, three-way valve 4 is turned to isolate probe circuit 102 from the sterile water supply bags 1 and vacuum pump 5 is shut off. This effectively creates the liquid system as shown in FIG. 5.

Peristaltic pump 20 is subsequently activated to circulate sterile degassed water from reservoir container 8 through the tubing 108, 104, 10, and 126 into the probe head 11 and back to the rigid reservoir container via tubing 106. As the liquid is pumped, air is displaced from all of the elements and flows into reservoir container 8 and in turn rises into the overflow bag through vent line 124.

Once all of the air is expelled from probe head 11 and hydraulic circuit 102, shut-off valves 15 and 15a are turned or pinched to isolate overflow bag 6. The overflow bag is removed from the tubing.

Syringe 12 is attached to overflow bag 6 after a plunger 134 of the syringe is pushed all the way in. Overflow bag 6 is positioned such that the air is at the top and the liquid is next to the syringe connection. The syringe plunger 134 may then be retracted to fill or partially fill the syringe 12 with sterile degassed liquid without entrained air. Syringe 12 is then removed from bag 6 and attached to hydraulic circuit 102 and particularly to tube 106 via three-way valve 13. Three-way valve 13 is turned to allow liquid to flow from syringe 12 into probe outlet tube 106.

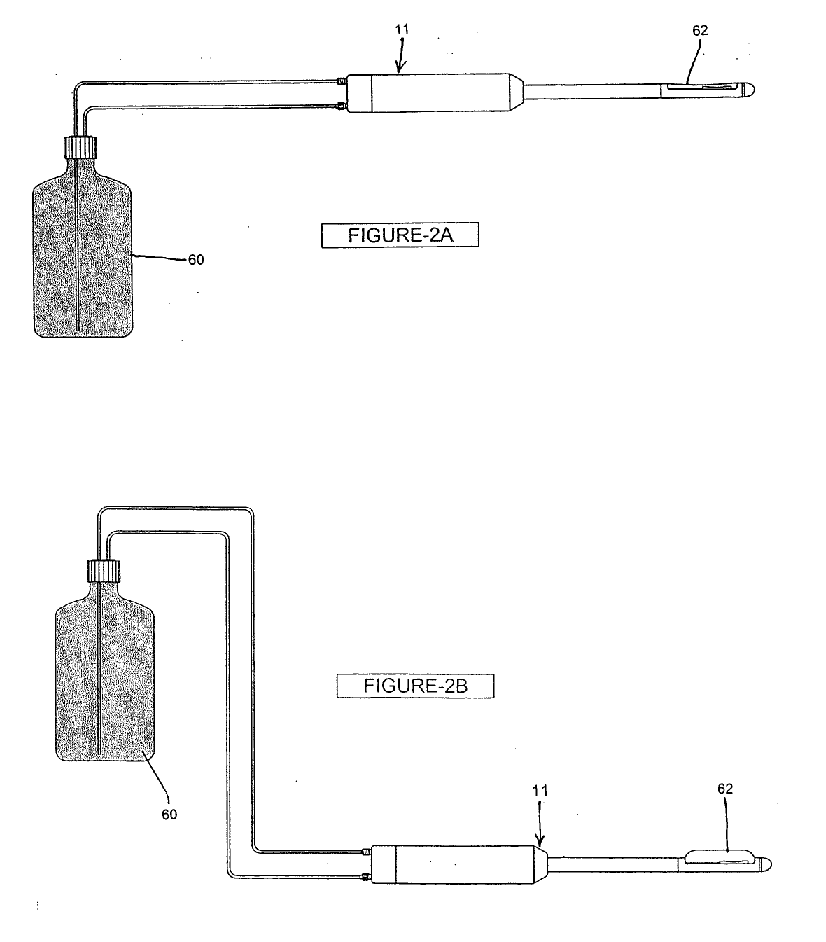

At this point, an air free, degassed and sterile liquid system exists, as shown in FIG. 6. If the peristaltic pump 20 is left on, the degassed sterile medical irrigation water will be circulated through the system and particularly through probe 11. Since the liquid system is free of compressible air and closed to the atmosphere, probe 11 may be disposed at any height relative to reservoir container 8 without causing the liquid pressure to change. This keeps the height or degree of distension of a bolus 62 (see FIG. 2B) constant.

If the bolus height is to be adjusted, the syringe plunger 134 may be moved in and out. The water in syringe 12 will serve to pressurize the liquid system. Since the bolus 62 is flexible, it will expand or contract as the static pressure of the system rises above the ambient air pressure. Adjusting this pressure differential with the syringe plunger 134 easily sets the amount the bolus 62 expands.

The HIFU system may then be used as per its specifications.

In this manner, a sterile, degassed supply of liquid may be manufactured on site, at relatively low cost and the tubing sets may be presterilized and disposable, reducing time and cost of the end user.

In practice, the tubing described must be manufactured with a medical grade polymer. Such polymers generally have a high surface tension that can serve to trap air bubbles or cause them to stick to the internal surfaces of the tube. This tendency can be eliminated, if desired, by coating all internal surfaces of the tubing runs and the internal surfaces of the probe assembly with an agent that reduces said surface tension and serves to effectively lubricate the surfaces to allow for quicker bubble expulsion. One such agent consists of cross-linked polymers that bond to the parent plastic and reduce surface friction or tension of the tubing. Other commercially available products can be used with equal success. This element is not mandatory to achieve the desired objectives of the invention but can serve to provide a shorter time to degas and set up a system.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method for use in preparation for a medical procedure, comprising:
providing a medical instrument;
feeding sterile water to a reservoir container;
during the feeding of said water to said reservoir container, degassing the water;
upon a filling of the reservoir container with the degassed sterile water, operatively connecting said reservoir to said medical instrument in a hydraulic circuit;
subsequently pumping degassed sterile water from said reservoir container through said circuit; and
during the pumping, removing air from said circuit, to enable the creation of a closed circuit, containing degassed sterile water and a substantial absence of air, to render said medical instrument in condition for a medical procedure.

2. The method defined in claim 1 wherein the degassing of the sterile water comprises feeding the sterile water through a degassing hydrophobic hollow fiber or membrane filter and, during the feeding of said water through said filter, operating a vacuum pump connected to said filter to extract dissolved gas from the water.

3. The method defined in claim 2, further comprising operatively disconnecting said filter from said reservoir container after the filling thereof.

4. The method defined in claim 3 wherein the disconnecting of said filter includes actuating a valve disposed between said filter and said reservoir container.

5. The method defined in claim 2, further comprising:
connecting said vacuum pump to said filter prior to the feeding of the sterile water through said filter; and
connecting a hydrophobic secondary filter between said vacuum pump and said filter.

6. The method defined in claim 1 wherein the feeding of sterile water to said reservoir container is carried out as a gravity feed operation.

7. The method defined in claim 6, further comprising:
connecting an overflow container to said reservoir container; and
conveying degassed sterile water from said reservoir container to said overflow container, said overflow container being disposed at a vertical location higher than a vertical location of said reservoir container and lower than a vertical location of a source of said sterile water.

8. The method defined in claim 7 wherein said overflow container is provided with an air outlet port impermeable to a passage of liquid water, further comprising connecting said overflow container to said reservoir container so as to enable a passage of air from said reservoir container to said overflow container and out of said overflow container through said outlet port.

9. The method defined in claim 1, further comprising connecting to said hydraulic circuit a syringe containing an amount of degassed sterile water, for enabling an increase in water content of said hydraulic circuit after the removing of air from said circuit and after a closing of said circuit.

10. The method defined in claim 9, further comprising:
connecting an overflow container to said reservoir container;
conveying degassed sterile water from said reservoir container to said overflow container; and
thereafter isolating said overflow container from said reservoir container and connecting said syringe to said overflow container to extract degassed sterile water from said container.

11. The method defined in claim 1 wherein said medical instrument is a high intensity focused ultrasound probe.

12. A method for use in preparation for a medical procedure, comprising:
providing a medical instrument;
connecting said instrument to a hydraulic circuit including a pump and a reservoir container;
filling said container with degassed sterile irrigation water;
removing air from said circuit; and
pumping degassed sterile irrigation water from said reservoir container through said instrument and back to said reservoir container.

13. The method defined in claim 12 wherein the filling of said reservoir container includes:
feeding sterile water to a reservoir container; and
during the feeding of said water to said reservoir container, degassing the water.

14. The method defined in claim 13 wherein the degassing of the sterile water comprises feeding the sterile water through a degassing hydrophobic hollow fiber or membrane filter and, during the feeding of said water through said filter, operating a vacuum pump connected to said membrane filter to extract dissolved gas from the water.

15. The method defined in claim 12 wherein the feeding of sterile water to said reservoir container is carried out as a gravity feed operation.

16. The method defined in claim 16, further comprising:
connecting an overflow container to said reservoir container; and
conveying degassed sterile water from said reservoir container to said overflow container,
said overflow container being disposed at a vertical location higher than a vertical location of said reservoir container and lower than a vertical location of a source of said sterile water.

17. The method defined in claim 12, further comprising connecting to said hydraulic circuit a syringe containing an amount of degassed sterile water, for enabling an increase in water content of said hydraulic circuit after the removing of air from said circuit and after a closing of said circuit.

18. A kit for use in preparation for a medical procedure utilizing a medical instrument, said kit comprising:
a hydrophobic hollow fiber or membrane filter;
a reservoir container;
a plurality of valves; and
tubing for constructing a first hydraulic circuit including the filter and the reservoir container and a second hydraulic circuit including the reservoir container and the medical instrument, for connecting an input end of the filter to a source of sterile medical water in the first hydraulic circuit, for connecting a pump in the second hydraulic circuit between said reservoir container and the medical instrument, for coupling said filter to a vacuum pump to degas water passing through said filter in the first hydraulic circuit, and for enabling a release of air from the second hydraulic circuit during an operation of said pump circulating degassed sterile water from said reservoir container to said medical instrument and back to said reservoir container,
said valves being disposed in said first hydraulic circuit for facilitating an operative connecting of said filter to said reservoir container to enable flow of degassed sterile water from said filter to said reservoir container and simultaneously an isolating of said medical instrument from said reservoir container and said filter and for enabling a subsequent isolation of said filter from said reservoir container while permitting fluid communication between said reservoir container and the medical instrument in said second hydraulic circuit.

19. The kit defined in claim 17, further comprising an overflow container connectable to said reservoir container via said tubing.

20. The kit defined in claim 18 wherein said overflow container is provided with an air outlet port impermeable to a passage of liquid water, said overflow container being connectable to said reservoir container so as to enable a passage of air from said reservoir container to said overflow container and out of said overflow container through said outlet port.

21. The kit defined in claim 17, further comprising a syringe connectable to said reservoir container for holding an amount of degassed sterile water to enable an increase in water content of said second hydraulic circuit after a removing of air from said second hydraulic circuit and after a closing of said second hydraulic circuit.

22. A hydraulic circuit assembly for use in preparing for and carrying out a medical therapeutic method using a medical instrument, comprising:
a hydrophobic hollow fiber or membrane filter connectable on an upstream side to a source of sterile medical water and also connectable to a vacuum pump for degassing sterile water flowing through the filter;
a three-way valve;
a reservoir container operatively coupled to an outlet of said filter via said three-way valve;
a pump operatively linked on an upstream side to said three-way valve and on a downstream side to an inlet of the medical instrument, said probe having an outlet operatively connected to said reservoir container; and
at least one valve or outlet port connected to said reservoir container for enabling a removal of air from said reservoir container and from a subcircuit including said reservoir container and the medical instrument.

23. The circuit assembly defined in claim 21, further comprising an overflow container operatively connected to said reservoir container for receiving degassed sterile water therefrom.

24. The circuit assembly defined in claim 22 wherein said at least one valve or outlet port is provided on said overflow container.

25. The circuit assembly defined in claim 21, further comprising a syringe connectable to said subcircuit for containing an amount of degassed sterile water to enable an increase in water content of said subcircuit after a removing of air from said subcircuit and after a closing of said subcircuit.
